(19) United States
(12) Patent Application Publication
(10) Pub. No.: US 2026/0174432 A1
(43) Pub. Date: Jun. 25, 2026

(54) DEVICES, SYSTEMS, AND METHODS FOR PERFORMING A SURGICAL PROCEDURE

(71) Applicant: Glaive, LLC, Westfield, IN (US)

(72) Inventors: Nathan Graves, Westfield, IN (US); Nathan Glass, Westfield, IN (US)

(73) Assignee: Glaive, LLC, Westfield, IN (US)

(21) Appl. No.: 19/125,128

(22) PCT Filed: Nov. 13, 2023
(86) PCT No.: PCT/US2023/037205

§ 371 Date: Apr. 28, 2025

Related U.S. Application Data
(60) Provisional application No. 63/424,231, filed on Nov. 10, 2022.

(51) Int. Cl. A61B 17/068; A61B 17/02; A61B 17/16; A61B 17/3207

(52) U.S. Cl. CPC A61B 17/068; A61B 17/0218; A61B 17/1617; A61B 17/1637; A61B 17/1671; A61B 17/320708; A61B 2017/320733

(57) ABSTRACT

Devices, systems, and methods for performing a surgical procedure. The present disclosure includes disclosure of a system, comprising a cannulated trocar comprising a trocar handle and defining a slot therein, a cutter comprising a cutting blade at a distal end of a blade shaft and a blade clamp configured to clamp the cutter to a surgical scope, and an elongated shim comprising a shim handle, the elongated shim configured to fit within a proximal aperture of the cannulated trocar and below the cutter so to cause the cutting blade to be exposed from the slot when positioned within the proximal aperture.

### PRIORITY

The present application is related to, and claims the priority benefit of, U.S. patent application Ser. No. 63/424,231, filed Nov. 10, 2022, the contents of which are incorporated herein directly and by reference in their entirety.

### BRIEF SUMMARY

The present disclosure includes disclosure of devices, systems, and methods for performing a surgical procedure.

The present disclosure includes disclosure of a cutter, a cannulated trocar, and a shim, as shown and/or described herein.

The present disclosure includes disclosure of a method of performing a surgical procedure, including but not limited to a gastrocnemius recession, as shown and/or described herein.

The present disclosure includes disclosure of a system, comprising an elongated cannulated trocar comprising a trocar handle and defining a slot at a relative top of the elongated cannulated trocar, a cutter comprising a cutting blade at a distal end of a blade shaft and a blade clamp configured to clamp the cutter to a surgical scope, and an elongated shim comprising a shim handle, the elongated shim configured to fit within a proximal aperture of the elongated cannulated trocar and below the cutter so to cause the cutting blade to be exposed from the slot.

The present disclosure includes disclosure of a system, comprising a cannulated trocar comprising a trocar handle and defining a slot therein, a cutter comprising a cutting blade at a distal end of a blade shaft and a blade clamp configured to clamp the cutter to a surgical scope, and an elongated shim comprising a shim handle, the elongated shim configured to fit within a proximal aperture of the cannulated trocar and below the cutter so to cause the cutting blade to be exposed from the slot when positioned within the proximal aperture.

The present disclosure includes disclosure of a system, wherein the cannulated trocar comprises an elongated body having the slot defined therein.

The present disclosure includes disclosure of a system, wherein the elongated body has a curvature.

The present disclosure includes disclosure of a system, wherein the curvature curves upward.

The present disclosure includes disclosure of a system, wherein the cutter comprises a blade shaft extending between the blade clamp and the cutting blade.

The present disclosure includes disclosure of a system, wherein the cutting blade has a blunt distal end.

The present disclosure includes disclosure of a system, wherein the cutting blade has a sharp proximal end.

The present disclosure includes disclosure of a system, wherein the elongated shim comprises an elongated shim body coupled to the shim handle.

The present disclosure includes disclosure of a system, wherein the elongated shim body is relatively flat with grooves defined therein.

The present disclosure includes disclosure of a system, wherein when the elongated shim is not positioned within the cannulated trocar along with the cutter, the cutting blade remains within the slot of the cannulated trocar.

The present disclosure includes disclosure of a method, comprising using the system of the present disclosure to perform a surgical procedure.

The present disclosure includes disclosure of a method, wherein the surgical procedure comprises a gastrocnemius recession.

The present disclosure includes disclosure of a method, wherein the step of using the system comprises coupling a surgical scope to the blade clamp of the cutter, inserting a distal end of the cutter into the proximal aperture of the cannulated trocar that has been positioned within a mammalian body, and advancing the distal end of the cutter to a tissue of interest within the mammalian body.

The present disclosure includes disclosure of a method, further comprising inserting the shim into the cannulated trocar to cause the cutting blade to extend through the slot of the cannulated trocar.

The present disclosure includes disclosure of a method, further comprising retracting the cutter so to cut the tissue of interest.

### BRIEF DESCRIPTION OF THE DRAWINGS

The disclosed embodiments and other features, advantages, and disclosures contained herein, and the matter of attaining them, will become apparent and the present disclosure will be better understood by reference to the following description of various exemplary embodiments of the present disclosure taken in conjunction with the accompanying drawings, wherein:

FIG. 1 shows individual components of a surgical system, according to an exemplary embodiment of the present disclosure;

FIG. 2 shows components of a surgical system used in connection with a surgical scope to perform a surgical procedure, according to an exemplary embodiment of the present disclosure;

FIG. 3 shows a cannulated trocar having a round trocar handle, according to an exemplary embodiment of the present disclosure;

FIG. 4 shows a shim having a rectangular handle, according to an exemplary embodiment of the present disclosure;

FIG. 5 shows a shim having a cylindrical handle, according to an exemplary embodiment of the present disclosure;

FIG. 6 shows a perspective view of a scope being used in connection with a cutter, according to an exemplary embodiment of the present disclosure;

FIG. 7 shows a side view of a scope being used in connection with a cutter, according to an exemplary embodiment of the present disclosure;

FIG. 8 shows a perspective view of a cutter, according to an exemplary embodiment of the present disclosure; and

FIG. 9 shows a side view of a cutter, according to an exemplary embodiment of the present disclosure.

As such, an overview of the features, functions and/or configurations of the components depicted in the various figures will now be presented. It should be appreciated that not all of the features of the components of the figures are necessarily described and some of these non-discussed features (as well as discussed features) are inherent from the figures themselves. Other non-discussed features may be inherent in component geometry and/or configuration. Furthermore, wherever feasible and convenient, like reference numerals are used in the figures and the description to refer to the same or like parts or steps. The figures are in a simplified form and not to precise scale.

### DETAILED DESCRIPTION

For the purposes of promoting an understanding of the principles of the present disclosure, reference will now be made to the embodiments illustrated in the drawings, and specific language will be used to describe the same. It will nevertheless be understood that no limitation of the scope of this disclosure is thereby intended.

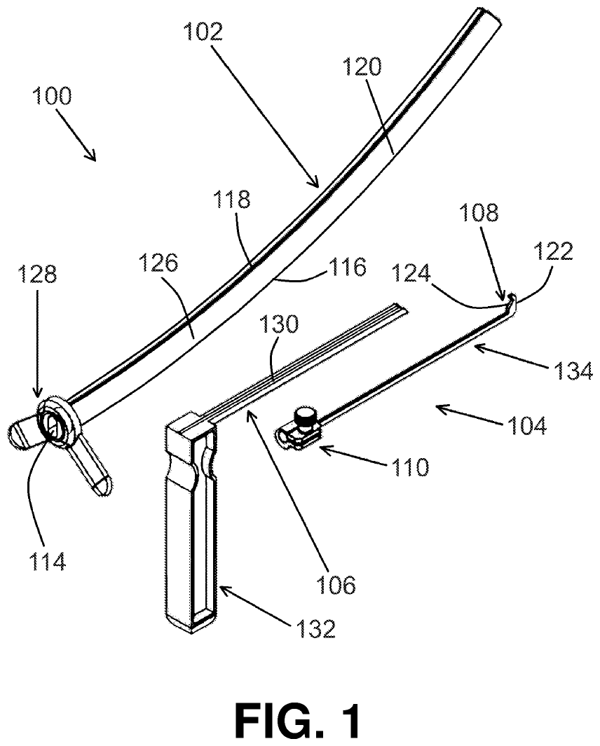

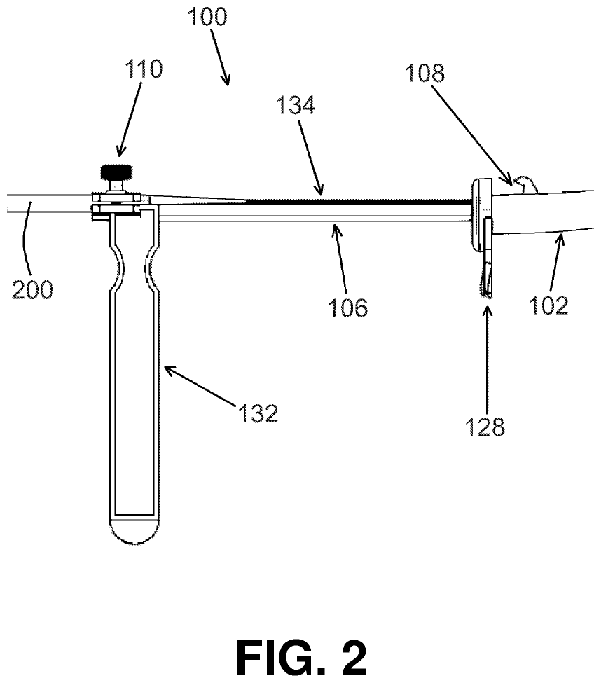

The present disclosure includes disclosure of a cannulated surgical instrument used for minimally invasive procedures, including, but not limited to, cutting tissue. An exemplary system 100 of the present disclosure, such as shown in FIG. 1, comprises a cannulated trocar 102, a cutter 104, and an elongated shim 106 used to unsheathe the cutting blade 108 of the cutter 104. When using a surgical scope 200 (shown in FIG. 2) to identify a desired location within the body, a blade clamp 110 of the cutter 104 can be connected to the surgical scope 200 such that the cutter 104 moves along with the surgical scope 200 when controlled by a user.

Procedurally, a cannulated trocar 102 is ultimately positioned at least partially within a mammalian body so that a relative distal end is at or near a desired location within the body. A surgical scope 200 can be passed through a first lumen 114 of the cannulated trocar 102 so that a user of the scope 200 has a visual depiction within the body at or near the desired location. The cutter 104 would be coupled to the surgical scope 200 so that the cutting blade 108 is at or near a relative distal end of the surgical scope 200. Movement of the cutter 104 within the cannulated trocar 102 would not cause the cutting blade 108 to cut any tissue so long as the shim 106 is not operatively used with such a procedure, as the cutting blade 108 would slide along a relative curved bottom 116 of the cannulated trocar 102 and not be exposed from a slot 118 defined along an elongated portion of the cannulated trocar 102. Insertion of the shim 106 into the cannulated trocar 102, below the cutter 104, would cause the cutter 104 to raise away from a relative internal bottom 120 of the cannulated trocar 102, causing the cutting blade 108 to be exposed through the slot 118. The curvature of the cannulated trocar 102 is also useful as it would better correspond to a shape of tissue to be cut, such as in the case of a gastrocnemius recession.

A distal portion 122 of the cutting blade 108 can be blunt so as to not be able to cut any tissue, while a proximal portion 124 of the cutting blade 108 is sharp, allowing for tissue to be cut. When the shim 106 is used in connection with the cutter 104 and the cannulated trocar 102, the cutter 104 can be retracted by retracting the surgical scope 200 coupled thereto, and if tissue is positioned at the proximal portion of the cutting blade 108, said tissue would be cut while the cutter 104 is retracted. So to permit effective use of the cutter 104, the surgical scope 200 would be flexible or semirigid.

As shown in FIG. 1, an exemplary cannulated trocar 102 of the present disclosure comprises an elongated body 126 having a curvature, a slot 118 defined therein along the elongated body 126, and a handle 128 at a relative proximal end for the user to engage. A first lumen 114 can be defined through the elongated body 126 of the cannulated trocar 102 as referenced herein.

An exemplary shim 106 of the present disclosure comprises an elongated shim body 130 coupled to a shim handle 132, such as shown in FIG. 1.

An exemplary cutter 104 of the present disclosure, such as shown in FIG. 1, comprises a cutting blade 108 having a blunt distal end 122 and a sharp proximal end 124, said cutting blade at a distal end of a blade shaft 134, with the blade clamp 110 at a proximal end of cutter 104.

FIG. 2 shows components of a surgical system 100 used in connection with a surgical scope 200 to perform a surgical procedure, according to an exemplary embodiment of the present disclosure. Various elements identified in FIG. 1 are also identified in FIG. 2.

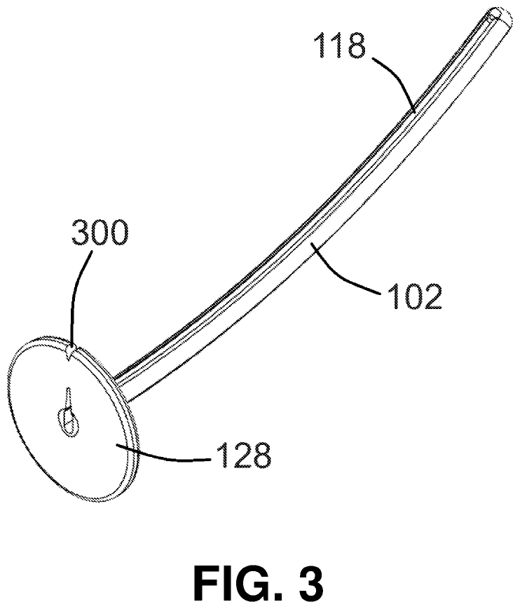

FIG. 3 shows a cannulated trocar 102 having a round trocar handle 128, according to an exemplary embodiment of the present disclosure. As shown therein, trocar handle 128 can have an alignment indicator 300, such as a notch, a marking, or another indicator suitable for indicating orientation of said cannulated trocar 102.

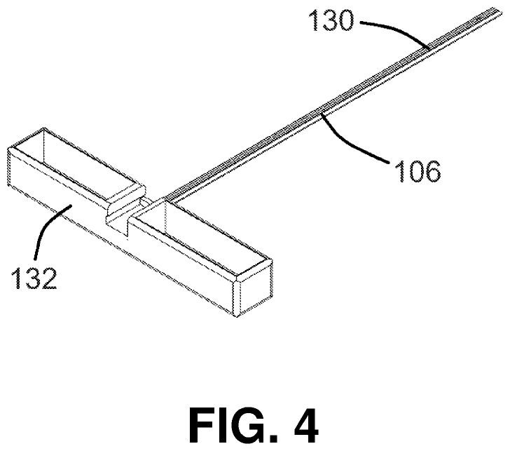

FIG. 4 shows a shim 106 having a rectangular handle 132, according to an exemplary embodiment of the present disclosure.

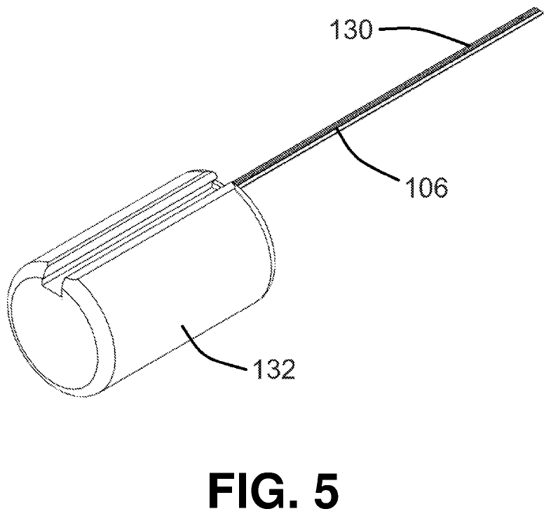

FIG. 5 shows a shim 106 having a cylindrical handle 132, according to an exemplary embodiment of the present disclosure.

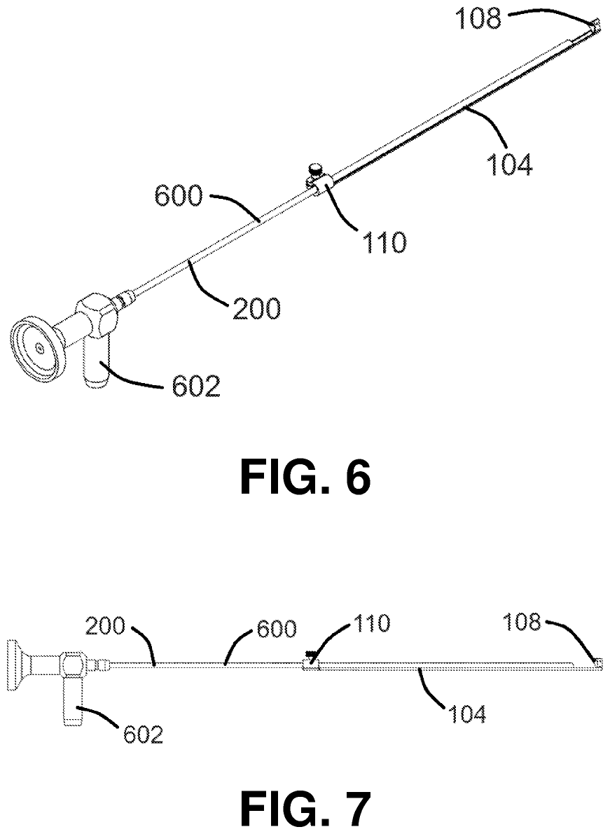

FIG. 6 shows a perspective view of a scope 200 being used in connection with a cutter 104, according to an exemplary embodiment of the present disclosure. As shown therein, scope 200 comprises an elongated scope body 600 configured to fit within blade clamp 110, and a scope handle 602 for engagement by a user.

FIG. 7 shows a side view of a scope 200 being used in connection with a cutter 104, according to an exemplary embodiment of the present disclosure.

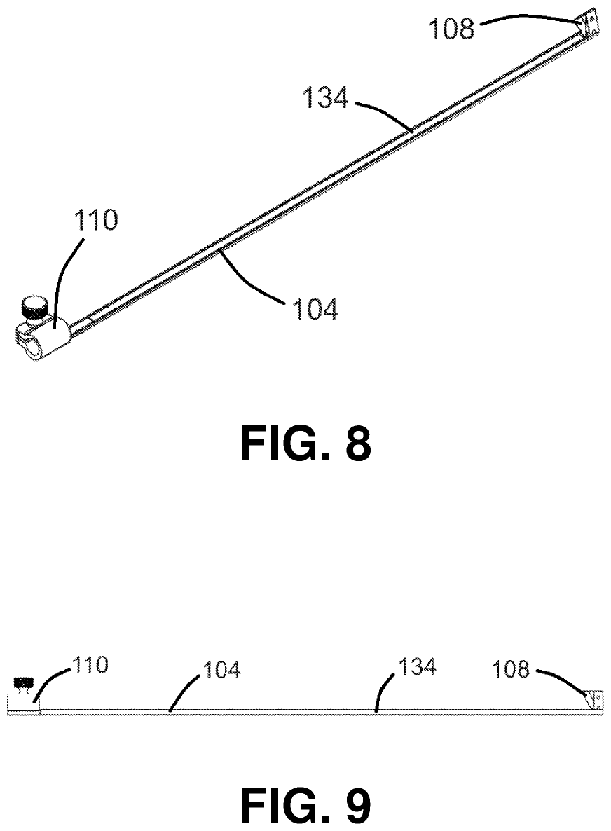

FIG. 8 shows a perspective view of a cutter, according to an exemplary embodiment of the present disclosure.

FIG. 9 shows a side view of a cutter, according to an exemplary embodiment of the present disclosure.

The present disclosure includes disclosure of a system 100, comprising a cannulated trocar 102 comprising a trocar handle 128 and defining a slot 118 therein, a cutter 104 comprising a cutting blade 108 at a distal end of a blade shaft 134 and a blade clamp 110 configured to clamp the cutter 104 to a surgical scope 200, and an elongated shim 106 comprising a shim handle 132, a portion of the elongated shim 106 configured to fit within a proximal aperture 114 of the cannulated trocar 102 and below the cutter 104 so to cause the cutting blade 108 to be exposed from the slot 118 when positioned therein.

The present disclosure includes disclosure of a system 100, wherein the cannulated trocar 102 comprises an elongated body 126 having the slot 118 defined therein.

The present disclosure includes disclosure of a system 100, wherein the elongated body 126 has a curvature.

The present disclosure includes disclosure of a system 100, wherein the curvature curves upward.

The present disclosure includes disclosure of a system 100, wherein the cutter 104 comprises a blade shaft 134 extending between the blade clamp 110 and the cutting blade 108.

The present disclosure includes disclosure of a system 100, wherein the cutting blade 108 has a blunt distal end 122.

The present disclosure includes disclosure of a system 100, wherein the cutting blade has a sharp proximal end 124.

The present disclosure includes disclosure of a system 100, wherein the elongated shim 106 comprises an elongated shim body 130 coupled to the shim handle 132.

The present disclosure includes disclosure of a system 100, wherein the elongated shim body 130 is relatively flat with grooves defined therein.

The present disclosure includes disclosure of a system 100, wherein when the elongated shim 106 is not positioned within the cannulated trocar 102 along with the cutter 104, the cutting blade 108 remains within the slot 118 of the cannulated trocar 102.

The present disclosure includes disclosure of a method, comprising using an exemplary system 100 of the present disclosure to perform a surgical procedure.

The present disclosure includes disclosure of a method, wherein the surgical procedure comprises a gastrocnemius recession.

The present disclosure includes disclosure of a method, wherein the step of using the system comprises coupling a surgical scope 200 to the blade clamp 110 of the cutter, inserting a distal end of the cutter 104 into the proximal aperture 114 of the cannulated trocar 102 that has been positioned within a mammalian body, and advancing the distal end of the cutter 104 to a tissue of interest within the mammalian body.

The present disclosure includes disclosure of a method, further comprising inserting the shim 106 into the cannulated trocar 102 to cause the cutting blade 108 to extend through the slot 118 of the cannulated trocar.

The present disclosure includes disclosure of a method, further comprising retracting the cutter 104 so that the cutting blade can cut the tissue of interest.

While various embodiments of devices, systems, and methods have been described in considerable detail herein, the embodiments are merely offered as non-limiting examples of the disclosure described herein. It will therefore be understood that various changes and modifications may be made, and equivalents may be substituted for elements thereof, without departing from the scope of the present disclosure. The present disclosure is not intended to be exhaustive or limiting with respect to the content thereof.

Further, in describing representative embodiments, the present disclosure may have presented a method and/or a process as a particular sequence of steps. However, to the extent that the method or process does not rely on the particular order of steps set forth therein, the method or process should not be limited to the particular sequence of steps described, as other sequences of steps may be possible. Therefore, the particular order of the steps disclosed herein should not be construed as limitations of the present disclosure. In addition, disclosure directed to a method and/or process should not be limited to the performance of their steps in the order written. Such sequences may be varied and still remain within the scope of the present disclosure.

1. A system, comprising:
a cannulated trocar comprising a trocar handle and defining a slot therein;
a cutter comprising a cutting blade at a distal end of a blade shaft and a blade clamp configured to clamp the cutter to a surgical scope; and
an elongated shim comprising a shim handle, the elongated shim configured to fit within a proximal aperture of the cannulated trocar and below the cutter so to cause the cutting blade to be exposed from the slot when positioned therein.

2. The system of claim 1, wherein the cannulated trocar comprises an elongated body having the slot defined therein.

3. The system of claim 1, wherein the elongated body has a curvature.

4. The system of claim 3, wherein the curvature curves upward.

5. The system of claim 1, wherein the blade shaft extends between the blade clamp and the cutting blade.

6. The system of claim 5, wherein the cutting blade has a blunt distal end.

7. The system of claim 6, wherein the cutting blade has a sharp proximal end.

8. The system of claim 1, wherein the elongated shim comprises an elongated shim body coupled to the shim handle.

9. The system of claim 8, wherein the elongated shim body is relatively flat with grooves defined therein.

10. The system of claim 1, wherein when the elongated shim is not positioned within the cannulated trocar along with the cutter, the cutting blade remains within the slot of the cannulated trocar.

11. A method, comprising:
using the system of claim 1 to perform a surgical procedure.

12. The method of claim 11, wherein the surgical procedure comprises a gastrocnemius recession.

13. The method of claim 11, wherein the step of using the system comprises:
coupling a surgical scope to the blade clamp of the cutter;
inserting a distal end of the cutter into the proximal aperture of the cannulated trocar that has been positioned within a mammalian body; and
advancing the distal end of the cutter to a tissue of interest within the mammalian body.

14. The method of claim 13, further comprising:
inserting the shim into the cannulated trocar to cause the cutting blade to extend through the slot of the cannulated trocar.

15. The method of claim 14, further comprising:
retracting the cutter so that the cutting blade can cut the tissue of interest.
